SEQUENCE LISTING (69 sequences)

SEQ ID NO: 1 - protein, 72 aa, Homo sapiens
TSEGCQIIHPPWEGGIRYRGLTRDQVKAINFLPVDYEIEYVCRGEREVVGPKVRKCLANGSWTDMDTPSRCV

SEQ ID NO: 2 - protein, 33 aa, Homo sapiens
NVDSADAEEDDSDVWWGGADTDYADGSEDKVVE

SEQ ID NO: 3 - protein, 17 aa, synthetic construct
DDSDVWWGGADTDYADG

SEQ ID NO: 4 - protein, 9 aa, synthetic construct
DDSDVWWGG

SEQ ID NO: 5 - protein, 9 aa, synthetic construct
ADSDVWWGG

SEQ ID NO: 6 - protein, 9 aa, synthetic construct
DASDVWWGG

SEQ ID NO: 7 - protein, 9 aa, synthetic construct
DDADVWWGG

SEQ ID NO: 8 - protein, 9 aa, synthetic construct
DDSAVWWGG

SEQ ID NO: 9 - protein, 9 aa, synthetic construct
DDSDAWWGG

SEQ ID NO: 10 - protein, 9 aa, synthetic construct
DDSDVAWGG

SEQ ID NO: 11 - protein, 9 aa, synthetic construct
DDSDVWAGG

SEQ ID NO: 12 - protein, 9 aa, synthetic construct
AASDVWWGG

SEQ ID NO: 13 - protein, 9 aa, synthetic construct
DASAVWWGG

SEQ ID NO: 14 - protein, 9 aa, synthetic construct
ADSAVWWGG

SEQ ID NO: 15 - protein, 9 aa, synthetic construct
  SITE 1: note = D-aspartic acid
DDSDVWWGG

SEQ ID NO: 16 - protein, 9 aa, synthetic construct
  SITE 2: note = D-aspartic acid
DDSDVWWGG

SEQ ID NO: 17 - protein, 9 aa, synthetic construct
  SITE 3: note = D-serine
DDSDVWWGG

SEQ ID NO: 18 - protein, 9 aa, synthetic construct
  SITE 4: note = D-aspartic acid
DDSDVWWGG

SEQ ID NO: 19 - protein, 9 aa, synthetic construct
  SITE 1..2: note = D-aspartic acid
  SITE 3: note = D-serine
DDSDVWWGG

SEQ ID NO: 20 - protein, 8 aa, synthetic construct
DDSDVWWG

SEQ ID NO: 21 - protein, 5 aa, synthetic construct
DVWWG

SEQ ID NO: 22 - protein, 7 aa, synthetic construct
DDSDVWW

SEQ ID NO: 23 - protein, 5 aa, synthetic construct
DIWWS

SEQ ID NO: 24 - protein, 5 aa, synthetic construct
DVWWS

SEQ ID NO: 25 - protein, 5 aa, synthetic construct
DIWWG

SEQ ID NO: 26 - protein, 5 aa, synthetic construct
DIYWS

SEQ ID NO: 27 - protein, 5 aa, synthetic construct
DIHWS

SEQ ID NO: 28 - protein, 5 aa, synthetic construct
NIWWS

SEQ ID NO: 29 - protein, 5 aa, synthetic construct
DIFWS

SEQ ID NO: 30 - protein, 5 aa, synthetic construct
DIYWG

SEQ ID NO: 31 - protein, 5 aa, synthetic construct
NFYWS

SEQ ID NO: 32 - protein, 5 aa, synthetic construct
NIWWG

SEQ ID NO: 33 - protein, 5 aa, synthetic construct
DVYWG

SEQ ID NO: 34 - protein, 5 aa, synthetic construct
GVYWS

SEQ ID NO: 35 - protein, 5 aa, synthetic construct
NVWWS

SEQ ID NO: 36 - protein, 5 aa, synthetic construct
GIYWS

SEQ ID NO: 37 - protein, 5 aa, synthetic construct
DVFWS

SEQ ID NO: 38 - protein, 5 aa, synthetic construct
DVYWS

SEQ ID NO: 39 - protein, 5 aa, synthetic construct
DIHWG

SEQ ID NO: 40 - protein, 5 aa, synthetic construct
NVWWG

SEQ ID NO: 41 - protein, 5 aa, synthetic construct
SVYWS

SEQ ID NO: 42 - protein, 5 aa, synthetic construct
NIYWG

SEQ ID NO: 43 - protein, 5 aa, synthetic construct
DVHWS

SEQ ID NO: 44 - protein, 5 aa, synthetic construct
PIHWS

SEQ ID NO: 45 - protein, 5 aa, synthetic construct
NIYWS

SEQ ID NO: 46 - protein, 5 aa, synthetic construct
PIYWS

SEQ ID NO: 47 - protein, 5 aa, synthetic construct
DVFWG

SEQ ID NO: 48 - protein, 5 aa, synthetic construct
PVHMS

SEQ ID NO: 49 - protein, 5 aa, synthetic construct
  SITE 1: note = D-aspartic acid
  SITE 3: note = (isoethylmethyl)benzene
DIXWS

SEQ ID NO: 50 - protein, 5 aa, synthetic construct
  SITE 1: note = D-aspartic acid
  SITE 3: note = 6-chloro-3-methyl-1H-indole
DIXWS

SEQ ID NO: 51 - protein, 5 aa, synthetic construct
  SITE 1: note = D-aspartic acid
  SITE 3: note = methylcyclohexane
DIXWS

SEQ ID NO: 52 - protein, 5 aa, synthetic construct
  SITE 1: note = D-aspartic acid
  SITE 3: note = ethylcyclohexane
DIXWS

SEQ ID NO: 53 - protein, 5 aa, synthetic construct
  SITE 1: note = D-aspartic acid
  SITE 3: note = 2-naphtalene
DIXWS

SEQ ID NO: 54 - protein, 5 aa, synthetic construct
  SITE 1: note = D-aspartic acid
  SITE 3: note = ethylbenzene
DIXWS

SEQ ID NO: 55 - protein, 5 aa, synthetic construct
  SITE 1: note = D-aspartic acid
  SITE 3: note = 1,1-difluoro-4-cyclohexyl
DIXWS

SEQ ID NO: 56 - protein, 5 aa, synthetic construct
  SITE 1: note = D-aspartic acid
  SITE 3: note = 4-methyl-1-methoxy-2-methylbenzene
DIXWS

SEQ ID NO: 57 - protein, 5 aa, synthetic construct
  SITE 1: note = D-aspartic acid
  SITE 3: note = 1-chloro-4-methylbenzene
DIXWS

SEQ ID NO: 58 - protein, 5 aa, synthetic construct
  SITE 1: note = D-aspartic acid
  SITE 3: note = (4-methylphenyl)methanol
DIXWS

SEQ ID NO: 59 - protein, 5 aa, synthetic construct
  SITE 1: note = D-aspartic acid
  SITE 3: note = 3-methylbenzoic acid
DIXWS

SEQ ID NO: 60 - protein, 5 aa, synthetic construct
  SITE 1: note = D-aspartic acid
  SITE 3: note = 4-methylaniline
DIXWS

SEQ ID NO: 61 - protein, 5 aa, synthetic construct
  SITE 1: note = D-aspartic acid
  SITE 3: note = N-ethyl-tryptophan
DIXWS

SEQ ID NO: 62 - protein, 5 aa, synthetic construct
  SITE 1: note = D-aspartic acid
DIYWS

SEQ ID NO: 63 - protein, 5 aa, synthetic construct
  SITE 1: note = D-aspartic acid
  SITE 5: note = L-serine coupled to C-N-(1,3,4-thiadiazol-2-yl)amide
DIYWX

SEQ ID NO: 64 - protein, 5 aa, synthetic construct
  SITE 1: note = D-aspartic acid
  SITE 5: note = L-serine coupled to C-N-(2-(4-methylpiperazin-1-yl)-2-oxoethyl)amide
DIYWX

SEQ ID NO: 65 - protein, 5 aa, synthetic construct
  SITE 1: note = D-aspartic acid coupled to (1-aminocyclobutyl)N-methanone
XIYWS

SEQ ID NO: 66 - protein, 5 aa, synthetic construct
  SITE 1: note = D-aspartic acid
DIYYS

SEQ ID NO: 67 - protein, 4 aa, synthetic construct
IWWG

SEQ ID NO: 68 - protein, 4 aa, synthetic construct
IWWG

SEQ ID NO: 69 - protein, 4 aa, synthetic construct
DVWW

(19) United States
(12) Patent Application Publication
(10) Pub. No.: US 2024/0376149 A1
(43) Pub. Date: Nov. 14, 2024

(54) Novel Modulators of GABABR1a

(71) Applicants: VIB VZW, Gent (BE); Katholieke Universiteit Leuven, Leuven (BE); Vrije Universiteit Brussel, Brussel (BE)

(72) Inventors: Joris De Wit, Kessel-Lo (BE); Ana Rita Santos, Herent (BE); Joao Carvalho, Leuven (BE); Laurent Galibert, Leuven (BE); Inge Van Molle, Brussel (BE); Han Remaut, Roosbeek (BE); Sylvain Celanire, Leuven (BE)

(21) Appl. No.: 18/683,030

(22) PCT Filed: Aug. 16, 2022
(86) PCT No.: PCT/EP2022/072835

§ 371 Date: Feb. 12, 2024

(30) Foreign Application Priority Data
Aug. 13, 2021 (EP) 21191352.0

(51) Int. Cl. C07K 7/06; A61K 38/00; A61P 25/00

(52) U.S. Cl. CPC C07K 7/06; A61P 25/00; A61K 38/00

(57) ABSTRACT

The present invention relates to the field of disorders of the central and peripheral nervous system, in particular neurological and psychiatric disorders, and the prevention and/or treatment thereof. In particular, the present invention relates to the finding that short peptides derived from the soluble amyloid precursor protein α (sAPPα) bind and modulate GABA_{B}R1a. The peptides are provided for clinical use, more particularly for the treatment of neurological diseases such as CMT as well as for psychiatric disorders.

### CROSS REFERENCE TO RELATED APPLICATIONS

This application Is a national phase entry under 35 U.S.C. § 371 of International Patent Application PCT/EP2022/072835, filed Aug. 16, 2022, designating the United States of America and published in English as International Patent Publication WO 2023/017190 on Feb. 16, 2023, which claims the benefit under Article 8 of the Patent Cooperation Treaty to European Patent Application Serial No. 21191352.0, filed Aug. 13, 2021, the entireties of which are hereby incorporated by reference.

### INCORPORATION BY REFERENCE

The ST.26 XML Sequence listing named “10340US_SequenceListing”, created on Aug. 16, 2022, and having a size of 74,846 bytes, is hereby incorporated herein by this reference in its entirety.

### FIELD OF THE INVENTION

The present invention relates to the field of disorders of the central and peripheral nervous system, in particular neurological and psychiatric disorders, and the prevention and/or treatment thereof. In particular, the present invention relates to the finding that short peptides derived from the soluble amyloid precursor protein α (sAPPα) bind and modulate GABABR1a. The peptides are provided for clinical use, more particularly for the treatment of neurological diseases such as CMT as well as for psychiatric disorders.

### BACKGROUND

The GABA B receptor (GABABR or GABA_{B}R), the metabotropic receptor for the inhibitory neurotransmitter γ-aminobutyric acid (GABA), regulates presynaptic neurotransmitter release and postsynaptic membrane excitability (Gassmann & Bettler, 2012 Nat Rev Neurosci 13: 380-394). It consists of two subunits: GABABR1, which binds GABA, and GABABR2, which couples to G proteins (Pin & Bettler, 2016 Nature 540: 60-68). Two major isoforms, GABABR1a and GABABR1b, differ by two N-terminal sushi repeats only present in the a-variant (Pin & Bettler, 2016 Nature 540: 60-68). GABABR signalling has been implicated in a number of neurological and psychiatric disorders, including cognitive impairments, anxiety, depression, schizophrenia, epilepsy, obsessive compulsive disorder, addiction and pain (Calver et al 2002 Neurosignals 11; Bettler et al 2004 Physiol Rev 84: 835-867). Selective binding partners of the GABABR1a sushi domains are of potential therapeutic interest owing to localization and functional differences of GABABR1 isoforms (Vigot et al 2006 Neuron 50: 589-601; Foster et al 2013 Br J Pharmacol 168: 1808-1819) as well as the adverse effects of current nonspecific agonists (Pin & Bettler, 2016 Nature 540: 60-68). Hence, functional GABABR1a-specific binding partners are valuable targets for the development of therapeutic strategies for modulating GABABR1a-specific signalling in neurological and psychiatric disorders.

It was recently demonstrated that GABA_{B}R1a acts as a synaptic receptor for secreted amyloid-β precursor protein (sAPP) (Rice et al 2019 Science 363; WO2018015296A1). sAPP specifically interacts with the sushi domain 1 of GABA_{B}R1a via its extension domain and modulates hippocampal synaptic plasticity and neurotransmission in vivo, more particularly the interaction acts as an activity-dependent negative-feedback mechanism to suppress synaptic release and maintain proper homeostatic control of neural circuits. Interestingly, the number of sAPP amino acids crucial for the interaction with the sushi domain could be minimized. A 17-mer peptide exerted effects similar to those of the full length sAPP, while a 9 amino acid short peptide can still bind the GABABR1a sushi 1 domain (WO2018015296A1). Recently, modifications of the 9-mer based on in silico modelling were suggested by Feng et al (2021 Chem Sci 12).

### SUMMARY

In current application novel GABA_{B}R1a modulators are disclosed. More particularly, the inventors of current application developed variants of the wild-type sAPP 9-mer and truncated the 9-mer to functional 8-, 7- and 5-mers. Additionally, peptidomimetics of said 5-mers were developed with improved binding properties to the GABABR1a sushi domain 1 compared to the sAPP 17-mer.

The application provides a GABA_{B}R1a binding peptide comprising the sequence X₁X₂X₃X₄X₅, wherein X₁ can be D, N, G, P or S; X₂ can be V or I; X₃ can be W, F, Y or H; X₄ can be W or Y; and X₅ can G or S, or a peptidomimetic of said GABABR1a binding peptide. In one embodiment, the peptide has a length of between 5 and 9 amino acids and is not DDSDVWWGG. In another embodiment, the GABA_{B}R1a binding peptide or peptidomimetic thereof comprises a W on position X₄. In a particular embodiment, the GABA_{B}R1a binding peptide is selected from the list consisting of DVWWG, DVWWS, DIWWS, DIWWG, DIFWS, DIYWS, DIYWG, GVYWS, NIWWG, NVWWS and DVYWG. In another particular embodiment, the peptidomimetic of the GABA_{B}R1a binding peptide is provided, wherein X₁ is D, X₂ is I, X₄ is W, X₅ is S and wherein X₃ is selected from the list consisting of isoethylmethyl-benzene, 6-chloro-3-methyl-1H-indole, methylcyclohexane, ethylcyclohexane, 2-naphthalene, ethylbenzene, 1,1-difluoro-4-cyclohexyl, 4-methyl-1-methoxy-2-methylbenzene, 1-chloro-4-methylbenzene, 4-methylphenyl-methanol, 3-methylbenzoic acid and 4-methylaniline. In a more particular embodiment, the peptidomimetic is selected from the list consisting of VIB-0068911-001, VIB-0068894-001, VIB-0068905-001, VIB-0068903-001, VIB-0068895-001, VIB-0068902-001, VIB-0068910-001, VIB-0068907-001, VIB-0068870-001, VIB-0068906-001, VIB-0068914-001 and VIB-0068912-001.

Any of the GABA_{B}R1a binding peptides herein disclosed or peptidomimetics thereof are provided for use as a medicament. More particularly, for use to treat cognitive impairments, anxiety, depression, epilepsy, dystonia, CMT, neuropathic pain, narcolepsy, spasticity, diabetes, multiple sclerosis, rheumatoid arthritis or COVID-19. This is similar as saying the methods of treating said disorders are provided, comprising administering any of the GABA_{B}R1a binding peptides herein disclosed or peptidomimetics thereof to a subject in need thereof. Also provided are methods of modulating the activity of GABA_{B}R1a in a subject, comprising administering any of the GABA_{B}R1a binding peptides herein disclosed or peptidomimetics thereof to the subject.

### SHORT DESCRIPTION OF THE FIGURES

FIG. 1 shows a cross-titration of the GABABR1a sushi 1 domain (SD1) in complex with FITC-9mer peptide to determine the optimal concentrations to use in the assay.

FIG. 2 shows the inhibition of SD1-FITC-9mer in the presence of increasing concentrations of the sAPP 17- and 9-mer peptides. The addition of the unlabelled peptides (17-mer and 9-mer) in excess, leads to a decrease in the signal, inhibiting the complex formation between SD1-9mer. The IC₅₀ values obtained for the two tool compounds were in agreement with previous measurements (IC₅₀ 17-mer=1.7 μM; IC₅₀ 9-mer=11 μM)). Contrary, the sAPP 10-mer did not show any alteration of the signal, meaning that this peptide is unable to disrupt or inhibit the interaction of SD1-FITC-9mer.

FIG. 3 shows the competition between GABABR1a binding peptides of the application and the wild-type sAPP 9-mer measured by FACS. HEK cells expressing GABABR1a-SD1 were incubated with peptides of the application and the binding of 9-mer in the presence of the peptides was measured. The 9-mer peptide is biotinylated and detected with a commercial antibody.

FIG. 4 shows the improvement of binding efficiency of the peptidomimetics effort. The bright circles represent the 5 mers consisting of natural amino acids (e.g. VIB_29,34, 43) while the dark squares represent the 5-mer compounds comprising non-natural entities (e.g. VIB 0068894, VIB-0068895). Both the natural and non-natural 5-mers are able to bind the sushi1 domain with similar affinity compared to the 17- and 9-mer despite the smaller size.

FIG. 5A and FIG. 5B shows fEPSP measurements in acute mouse hippocampal slices. FIG. 5A shows the increased facilitation upon administering 1 μM sAPP 17-mer compared to 1 μM scrambled control peptide. FIG. 5B shows that preincubating the slices with the GABA_{B}R antagonist CGP-54626 (10 μM) blocked the 17-mer induced increase in facilitation.

FIG. 6 shows the increase in facilitation upon adding the 5-mer P34 (1 μM) as well as P29 (1 μM) compared to the scrambled control (scr 17-mer).

FIG. 7 shows the statistically significant increase in short-term facilitation upon administration of P43, P34 or P47 (all at 10 μM) compared to the scrambled negative control at 10 μM. The sAPP 17-mer was used as positive control.

FIG. 8 demonstrates that the previously observed increase in short-term facilitation upon administering the P34 or P43 5-mers (all at 10 μM) was not detected anymore when the hippocampal slices were preincubated with the GABA_{B}R antagonist CGP-54626.

### DETAILED DESCRIPTION

The Applicants of current application previously disclosed that soluble APP (sAPP) binds the GABA_{B}R1a Sushi 1 domain through a 9 amino acid short fragment (WO2018015296A1). Based on in silico predictions and in vitro results the 9-mer was further truncated to a functional 5-mer. A large series of peptides consisting of natural and non-natural amino acids were designed and tested. Surprisingly, a selection of these peptides was shown to bind to the GABA_{B}R1a as well as the 9-mer sAPP. Moreover, several of these peptides demonstrate ex vivo modulation of GABA_{B}R1a activity.

### GABABR1a Modulating Peptides

In a first aspect, a GABA_{B}R1a binding peptide is provided comprising or consisting of the sequence X₁X₂X₃X₄X₅, wherein X₁ can be D, N, G, P or S; X₂ can be V or I; X₃ can be W, F, Y or H; X₄ can be W or Y; and X₅ can G or S. More particularly, a GABA_{B}R1a binding peptide is provided comprising or consisting of the sequence X₁X₂X₃WX₅, wherein X₁ can be D, N, G, P or S; X₂ can be V or I; X₃ can be W, F, Y or H; and X₅ can G or S.

“GABA_{B}R1a” as used herein refers to the gamma-aminobutyric acid (GABA) type B receptor subunit 1a, more particularly to the human GABA B1a receptor with GenBank accession number AAC98508, even more particularly to the sushi domain 1 of the human GABA B1a receptor. The following synonyms are interchangeably used in current application: “GABABR1a”, “GABA_{B}R1a”, “GABA B1a receptor”, “GABAB receptor 1a”. Also “the sushi 1 domain of GABABR1a”, “the GABABR1a sushi 1 domain”, “the sushi 1 domain”, “the sushi domain 1”, “the sushi 1 protein”, “SD1” or “GABABR1a-SD1” are used interchangeably and refer to SEQ ID No. 1.

SEQ ID No. 1: Sushi domain 1 of the human GABA
B1a receptor:
TSEGCQIIHPPWEGGIRYRGLTRDQVKAINFLPVDYEIEYVCRGEREVV
GPKVRKCLANGSWTDMDTPSRCV

Also provided is a peptidomimetic of any of the herein disclosed GABA_{B}R1a binding peptides. “Peptidomimetic” as used herein refers to a non-natural peptide or peptide comprising at least one non-natural amino acid (explained in more detail below). Peptidomimetics provide an alternative source of potent and selective Protein-Protein Interaction (PPI) modulators and occupy the chemical gap between small molecules and biologics, such as antibodies.

“Amino acids” as used herein refer to the structural units (monomers) that make up proteins. They join together to form short polymer chains called peptides or longer chains called either polypeptides or proteins. These chains are linear and unbranched, with each amino acid residue within the chain attached to two neighbouring amino acids. Twenty amino acids encoded by the universal genetic code are naturally incorporated into polypeptides and are called proteinogenic or natural amino acids. Natural amino acids or naturally occurring amino acids are glycine (Gly or G), Alanine (Ala or A), Valine (Val or V), Leucine (Leu or L), Isoleucine (lie or 1), Methionine (Met or M), Proline (Pro or P), Phenylalanine (Phe or F), Tryptophan (Trp or W), Serine (Ser or S), Threonine (Thr or T), Asparagine (Asn or N), Glutamine (Gln or Q), Tyrosine (Tyr or Y), Cysteine (Cys or C), Lysine (Lys or K), Arginine (Arg or R), Histidine (His or H), Aspartic Acid (Asp or D) and Glutamic Acid (Glu or E).

All amino acids (except for glycine) have two different stereoisomers or mirror images of their structure. These are labelled L (left-handed) and D (right-handed) to distinguish the mirror images. L-amino acids occur in all proteins produced by animals, plants, fungi and bacteria. In the Fisher projection, the amine group of L-amino acids occurs on the left side. In contrast, in D-amino acids, the amine group occurs in the right side in the Fisher projection. D-Amino acids are only occasionally found in nature as residues in proteins. The amino acids that make up the proteins in mammals are all L-amino acids. Hence, naturally occurring human proteins or peptides do not comprise D-amino acids.

In one embodiment, any of the GABA_{B}R1a binding peptides or peptidomimetics thereof disclosed herein comprises at least one D-amino acid. In a particular embodiment, the at least one D-amino acid is a D-Aspartic acid or a D-Serine. In a most particular embodiment, any of the GABA_{B}R1a binding peptides or peptidomimetics thereof disclosed herein are provided wherein X₁ is a D-Aspartic acid.

Besides natural amino acids, non-natural or unnatural amino acids have been developed. Non-natural amino acids are so called because they are not found in natural polypeptide chains. They are not among the 20 amino acids attached to tRNAs in living cells used to polymerize proteins. Some unnatural amino acids do occur naturally, but most are chemically synthesized. They can for example be made through chemical modifications of natural amino acids, such as N-methyl amino acids (attachment of a methyl group to the nitrogen in the amino group), alpha-methyl amino acids (a methyl group replaces the hydrogen on the alpha carbon), beta-amino acids (addition of a second carbon between the amino group and carboxy groups), homo-amino acids (addition of a methylene group between the alpha carbon and the side group) or beta-homo-amino acids (addition of a second carbon between the amino and carboxy groups and the addition of a methylene group between the alpha carbon and the side group). Unnatural amino acids are valuable building blocks in the manufacture of a wide range of pharmaceuticals. Non-natural amino acids can exhibit biological activity as free acids and they can be incorporated into linear or cyclic peptides with biological activity.

Non-limiting examples of non-natural amino acids are isoethylmethyl-benzene (also referred herein as F72), 6-chloro-3-methyl-1H-indole (also referred herein as F126), methylcyclohexane (also referred herein as F15), ethylcyclohexane (also referred herein as F141), 2-naphthalene (also referred herein as F195), ethylbenzene (also referred herein as F70), 1,1-difluoro-4-cyclohexyl (also referred herein as F182), 4-methyl-1-methoxy-2-methylbenzene (also referred herein as F158), 1-chloro-4-methylbenzene (also referred herein as F86), 4-methylphenyl-methanol (also referred herein as F44), 3-methylbenzoic acid (also referred herein as F50), 4-methylaniline (also referred herein as F57) and N-ethyl-tryptophan (also referred herein as F149).

Also provided is a GABA_{B}R1a binding peptide comprising or consisting of the sequence X₁X₂X₃WX₅, wherein X₁ can be D, N, G, P or S; X₂ can be V or I; X₃ can be W, F, Y, H or a non-natural amino acid; and X₅ can be G or S.

Also provided is a GABA_{B}R1a binding peptide comprising or consisting of the sequence X₁X₂X₃WX₅, wherein X₁ can be D, N, G, P or S; X₂ can be V or I; X₃ can be W, F, Y or H; and X₅ can be G or S, or wherein X₁ is D, X₂ is I, X₅ is S and X₃ is a non-natural amino acid.

The herein disclosed GABA_{B}R1a binding peptides or peptidomimetics thereof are from here on referred to as any of the GABA_{B}R1a binding peptides of the application.

In one embodiment, any of the GABA_{B}R1a binding peptides of the application has a length of between 5 and 17 amino acids, between 5 and 14, between 5 and 12, between 5 and 9 amino acids, between 5 and 8 amino acids or between 5 and 7 amino acids. In a particular embodiment, the length of the peptide or peptidomimetic is 5, 6, 7, 8 or 9 amino acids. In another particular embodiment, the GABA_{B}R1a binding peptides of the application have a length of 30, 25, 20, 15, 10 or less amino acids.

In another embodiment, any of the GABA_{B}R1a binding peptides of the application is not DDSDVWWGG. In a particular embodiment, any of the GABA_{B}R1a binding peptides of the application does not comprise DDSDVDWWG. In another embodiment, any of the GABA_{B}R1a binding peptides of the application is not DVWWG. In a particular embodiment, any of the GABA_{B}R1a binding peptides of the application does not comprise DVWWG.

In another embodiment, any of the GABA_{B}R1a binding peptides of the application is provided wherein the non-natural amino acid is selected from the list consisting of isoethylmethyl-benzene, 6-chloro-3-methyl-1H-indole, methylcyclohexane, ethylcyclohexane, 2-naphthalene, ethylbenzene, 1,1-difluoro-4-cyclohexyl, 4-methyl-1-methoxy-2-methylbenzene, 1-chloro-4-methylbenzene, 4-methylphenyl-methanol, 3-methylbenzoic acid and 4-methylaniline. In a particular embodiment, the GABA_{B}R1a binding peptide comprises the sequence DIX₃WS, wherein X₃ is selected from the list consisting of isoethylmethyl-benzene, 6-chloro-3-methyl-1H-indole, methylcyclohexane, ethylcyclohexane, 2-naphthalene, ethylbenzene, 1,1-difluoro-4-cyclohexyl, 4-methyl-1-methoxy-2-methylbenzene, 1-chloro-4-methylbenzene, 4-methylphenyl-methanol, 3-methylbenzoic acid, N-ethyl-tryptophan and 4-methylaniline. In an even more particular embodiment, the GABA_{B}R1a binding peptide comprising the sequence DIX₃WS is selected from the list consisting of VIB-0068911-011, VIB-0068894-001, VIB-0068905-001, VIB-0068903-001, VIB-0068895-001, VIB-0068902-001, VIB-0068910-001, VIB-0068907-001, VIB-0068870-001, VIB-0068906-001, VIB-0068914-001 and VIB-0068912-001.

In another particular embodiment, the GABA_{B}R1a binding peptide comprising the sequence X₁X₂X₃WX₅ is selected from the list consisting of DVWWG, DVWWS, DIWWS, DIWWG, DIFWS, DIYWS, DIYWG, DIHWG, DIHWS, DVFWG, DVFWS, GVYWS, GIYWS, NIWWG, NIWWS, NVWWG, NVWWS, NIYWG, NIYWS, NFYWS, DVYWG, DVYWS, DVHWS, DIFWS, SVYWS, PIHWS, PIYWS, DDSDVWWG, DIYWS* with S* being serine coupled to C-N-(1,3,4-thiadiazol-2-yl)amide, dIYWS** with S** being serine coupled to (C-N-(2-(4-methylpiperazin-1-yl)-2-oxoethyl)amide and d being D-aspartic acid, *dIYWS with d* being D-aspartic acid coupled to (1-aminocyclobutyl)N-methanone, and dIYWS with d being D-aspartic acid.

In even more particular embodiments, the GABA_{B}R1a binding peptide comprising the sequence X₁X₂X₃WX₅ is selected from the list consisting of DVWWG, DVWWS, DIWWS, DIWWG, DIFWS, DIYWS, DIYWG, GVYWS, NIWWG, NVWWS and DVYWG.

In another aspect, a GABA_{B}R1a binding peptide is provided comprising or consisting of IWWG, IWWS, DVWW, DDSDVWW or dIYYS with d being D-aspartic acid. In one embodiment a peptidomimetics of said GABABR1a binding peptide is provided.

In another aspect, a GABA_{B}R1a binding peptide is provided comprising or consisting of the sequence selected from DASAVWWGG or AASDVWWGG. In one embodiment a peptidomimetics of said GABABR1a binding peptide is provided.

In another aspect, a GABA_{B}R1a binding peptide is provided comprising or consisting of the sequence DDSDVWWGG, wherein at least one amino acid residue is a D-amino acid (i.e. D-stereoisomer). In a particular embodiment, the at least one D-amino acid is a D-Aspartic acid or a D-Serine. In an even more particular embodiment, the GABA_{B}R1a binding peptide comprising or consisting of the sequence DDSDVWWGG is provided wherein at least one D residue (i.e. Aspartic acid) is a D-Aspartic acid and/or S is a D-Serine. In an even more particular embodiment, the GABA_{B}R1a binding peptide comprising or consisting of the sequence DDSDVWWGG is provided wherein at least two D residues (i.e. Aspartic acid) are D-Aspartic acid and/or S is a D-Serine. In an even more particular embodiment, the GABA_{B}R1a binding peptide comprising or consisting of the sequence DDSDVWWGG is provided wherein the three D residues (i.e. Aspartic acid) are D-Aspartic acid and/or S is a D-Serine. In a most particular embodiment, the GABA_{B}R1a binding peptide comprising or consisting of the sequence DDSDVWWGG comprising at least one D-amino acid (i.e. D-stereoisomer) is selected from the list consisting of dDSDVWWG, DdSDVWGG, DDsDVWWGG, DDSdVWWGG and ddsDVWWGG, wherein d refers to a D-Aspartic acid and s to a D-Serine. In one embodiment a peptidomimetics of said GABABR1a binding peptide comprising at least one D-stereoisomer is provided.

### Pharmaceutical Compositions

In another aspect, a pharmaceutical composition is provided comprising any of the GABABR1a binding peptides or peptidomimetics thereof herein disclosed. More particularly, the pharmaceutical composition is comprised of a pharmaceutically acceptable carrier and a pharmaceutically effective amount of any of the GABABR1a binding peptides or peptidomimetics thereof herein disclosed, or salt thereof. In one embodiment, a pharmaceutically acceptable carrier is a carrier that is relatively non-toxic and innocuous to a patient at concentrations consistent with effective activity of the active ingredient so that any side effects ascribable to the carrier do not impair the beneficial effects of the active ingredient. A pharmaceutically effective amount of any of the GABABR1a binding peptides or peptidomimetics thereof is that amount which produces a result or exerts an influence on the particular condition being treated. Any of the GABABR1a binding peptides or peptidomimetics thereof can be administered with pharmaceutically acceptable carriers well known in the art using any effective conventional dosage unit forms, including immediate, slow and timed release preparations.

The pharmaceutical compositions of this application may also be in the form of oil-in-water emulsions. The emulsions may also contain sweetening and flavoring agents. Oily suspensions may be formulated by suspending the active ingredient in a vegetable oil such as, for example, arachis oil, olive oil, sesame oil or coconut oil, or in a mineral oil such as liquid paraffin. The pharmaceutical compositions may be in the form of sterile injectable aqueous suspensions. Such suspensions may be formulated according to known methods using suitable dispersing or wetting agents and suspending agents, all well-known by the person skilled in the art. The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides.

In addition, fatty acids such as oleic acid can be used in the preparation of injectables. The compositions of the invention can also contain other conventional pharmaceutically acceptable compounding ingredients, generally referred to as carriers or diluents, as necessary or desired. The nature of additional ingredients and the need of adding those to the composition of the invention is within the knowledge of a skilled person in the relevant art. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51 (4), 166-171.

### Therapeutic Application of the GABA_{B}R1a Sushi 1 Domain Binders

GABABR signalling has been implicated in a number of neurological and psychiatric disorders, including cognitive impairments, anxiety, depression, schizophrenia, epilepsy, obsessive compulsive disorder, addiction, migraine and pain (Calver et al 2002 Neurosignals 11; Bettler et al 2004 Physiol Rev 84: 835-867; Garcia-Martin et al 2017 Headache: The Journal of Head and Face Pain 57:1118-1135). It has been previously shown that wild-type 17-mer and 9-mer fragments of sAPP bind the GABABR1a Sushi 1 domain and that the 17-mer acts as a positive allosteric modulator of GABABR1a agonists (Rice et al 2019 Science; WO2018015296A1). Given that GABA is integral to the release of inhibitory neurotransmitters which produce a calming effect, the herein disclosed GABABR1a binding peptides and peptidomimetics thereof are provided to play a role in reducing the symptoms of GABABR1a-mediated neurological and psychiatric disorders, more particularly to play a role in reducing cognitive impairments, anxiety, stress, fear, depression, schizophrenia, epilepsy, obsessive compulsive disorder, addiction, migraine and/or pain.

Current application describes that the herein disclosed GABABR1a binding peptides or peptidomimetics thereof are GABABR1a agonists. It is therefore envisaged that said GABABR1a binding peptides or peptidomimetics thereof will be for use in any disease or disorder for which GABABR1a agonists are prescribed. Non-limiting examples of GABAB agonist medicaments are Baclofen, Progabide, Phenibut and Lesogaberan.

Baclofen, sold under the brand name Lioresal among others, is a medication used to treat spastic movement disorders or muscle spasticity such as from a spinal cord injury, cerebral palsy, CMT or multiple sclerosis. Baclofen has also been used for the treatment of alcohol use disorder. Progabide is approved in France for either monotherapy or adjunctive use in the treatment of epilepsy. Phenibut, sold under the brand names Anvifen, Fenibut and Noofen among others, is used to treat anxiety, insomnia, and for treatment of asthenia, depression, alcoholism, alcohol withdrawal syndrome, post-traumatic stress disorder, stuttering, tics, vestibular disorders, Meniere's disease, dizziness, for the prevention of motion sickness, and for the prevention of anxiety before or after surgical procedures or painful diagnostic tests. Lesogaberan was developed for the treatment of gastroesophageal reflux disease (Bredenoord 2009 IDrugs 12:576-584).

Therefore, in another aspect, any of the herein disclosed GABA_{B}R1a binding peptides or peptidomimetics thereof or pharmaceutical compositions herein described is provided for use as a medicament. From the art and overview above it is clear that GABABR1a agonists, including the herein disclosed GABA_{B}R1a binding peptides or peptidomimetics thereof or pharmaceutical compositions herein described, can be used to induce a calming effect of overstimulated synaptic activity that occurs for example in epilepsy, anxiety, stress, fear, stuttering, tics, vestibular disorders. Said GABABR1a agonists can also be used as muscle relaxant and thus to treat muscle spasticity for example in Charcot-Marie Tooth disease (CMT), dystonia, multiple sclerosis (MS), cerebral palsy or spinal cord injury. The herein disclosed GABA_{B}R1a binding peptides or peptidomimetics thereof or pharmaceutical compositions herein described are also provided for use to treat or reduce the symptoms of neurological or psychiatric disorders. Non-limiting examples are depression, alcoholism, post-traumatic stress disorder, insomnia. Said GABABR1a binding peptides are also provided for use to treat or reduce the symptoms of balance disorders. Non-limiting examples are Meniere's disease, dizziness, motion sickness.

In a particular embodiment, the herein disclosed GABA_{B}R1a binding peptides or peptidomimetics thereof or pharmaceutical compositions herein described are provided for use to treat or reduce the symptoms of cognitive impairments, anxiety, stress, fear, depression, schizophrenia, epilepsy, dystonia, CMT, neuropathic pain, narcolepsy or muscle spasticity or any disorder that can be treated by a GABABR1a agonist. This is similar as saying that methods of treating or reducing the symptoms of cognitive impairments, anxiety, depression, epilepsy, dystonia, CMT, neuropathic pain, narcolepsy or muscle spasticity or any disorder that can be treated by a GABABR1a agonist in a subject are provided, comprising the step of administering to the subject an effective amount of any of the herein disclosed GABABR1a binding peptides or peptidomimetics thereof.

It has been demonstrated that insulin content and secretion are higher in rat pancreatic islets after treatment with GABAB receptor agonists (Ligon et al 2007 Diabetologia 50: 764-773). Moreover, activation of GABAB receptors has recently been shown to inhibit disease progression in mouse models of type 1 diabetes (T1D), multiple sclerosis, rheumatoid arthritis, and COVID-19 (Tian et al 2021 Biomedicines 9:43). The herein disclosed GABABR1a binding peptides or peptidomimetics thereof are also provided for use to treat diabetes, more particularly type 1 diabetes, but also multiple sclerosis, rheumatoid arthritis, and COVID-19.

It has also been reported that the amyloid-beta 42 (Abeta42) peptide, the well-known trigger of neurotoxicity and synaptic loss associated with Alzheimer's disease, binds the GABA_{B}R1a sushi 1 domain and competes for SD1 binding with the sAPP 9-mer (Mei et al 2022 Ac Chem Neurosci 13: 2048-2059). Given that some of the herein disclosed sAPP 5-mers bind SD1 with higher binding affinities than the 9-mer, it is anticipated that these 5-mers overcome or destroy the Abeta42-SD1 binding, hence overcoming part of the Abeta42 neurotoxicity. Thus, the herein disclosed GABA_{B}R1a binding peptides or peptidomimetics thereof are also provided for use to treat Alzheimer's disease and/or other amyloid beta related disorders. In a most particular embodiment, the GABA_{B}R1a binding peptides selected from the list consisting of DDSDVWWG, DIWWS, DIWWG, VIB_0068911, VIB_0068894, VIB_0068905, VIB_0068903, VIB_0068895, VIB_0068910, VIB_0068907 and VIB_0068904 are provided for use to treat Alzheimer's disease and/or other amyloid beta related disorders.

“Treatment” refers to any rate of reduction or retardation of the progress of the disease or disorder compared to the progress or expected progress of the disease or disorder when left untreated. More desirable, the treatment results in no or zero progress of the disease or disorder (i.e. “inhibition” or “inhibition of progression”) or even in any rate of regression of the already developed disease or disorder.

“Reduction” or “reducing” as used herein refers to a statistically significant reduction of effects in the presence of the GABA_{B}R1a binders of the application compared to the absence of the GABA_{B}R1a binders. More particularly, a statistically significant reduction upon administering the GABA_{B}R1a binding peptide or peptidomimetics thereof of the invention compared to a control situation wherein the GABA_{B}R1a binding peptide is not administered. In a particular embodiment, said statistically significant reduction is an at least 25%, 30%, 35%, 40%, 45% or 50% reduction compared to the control situation.

Also disclosed are methods of modulating GABABR1a activity in a subject, comprising administering to the subject an effective amount of any of the herein disclosed GABABR1a binding peptides or peptidomimetics thereof. In one embodiment, modulating is statistically significantly increasing or enhancing. In another embodiment, modulating is statistically significantly reducing or decreasing.

Also disclosed are methods of reducing synaptic activity in a subject or in a subset of neurons in a subject, comprising administering to the subject an effective amount of any of the herein disclosed GABABR1a binding peptides or peptidomimetics thereof.

Also disclosed are methods of enhancing or stimulating GABABR1a related inhibition of neurotransmitter release in a subject, comprising administering to the subject an effective amount of any of the herein disclosed GABABR1a binding peptides or peptidomimetics thereof.

### Detection of GABABR1a Modulation

The effectivity of GABA_{B}R1a binding peptides herein disclosed in modulating GABA_{B}R1a activity both in vitro, ex vivo or in vivo can also be tested by various well-known techniques. For example by making use of the acute hippocampal slices of mice as shown herein.

### Administration

GABA_{B}R1a modulation can be achieved through the creation of transgenic organisms expressing one of the GABA_{B}R1a binders or by administering said GABA_{B}R1a binders to the subject. Whether the effect is achieved by expressing the GABA_{B}R1a binders or by administering the binder is not vital to the invention, as long as said GABA_{B}R1a binder modulates the GABA_{B}R1a activity. GABA_{B}R1a binders can be expressed from recombinant circular or linear DNA plasmids using any suitable promoter. Suitable promoters for expressing these GABA_{B}R1a binders from a plasmid include, for example, the U6 or H1 RNA pol Ill promoter sequences and the cytomegalovirus promoter. Selection of other suitable promoters is within the skill in the art. Non-limiting examples are neuronal-specific promoters, glial cell specific promoters, the human synapsin 1 gene promoter, the Hb9 promotor or the promoters disclosed in U.S. Pat. No. 7,341,847B2.

The recombinant plasmids can also comprise inducible or regulatable promoters for expression of the GABA_{B}R1a binders in a particular tissue or in a particular intracellular environment. The GABA_{B}R1a binders expressed from recombinant plasmids can either be isolated from cultured cell expression systems by standard techniques, or can be expressed intracellularly, e.g. in brain tissue or in neurons. GABA_{B}R1a binders can also be expressed intracellularly from recombinant viral vectors. The recombinant viral vectors comprise sequences encoding the GABA_{B}R1a binders of the invention and any suitable promoter for expressing them. The GABA_{B}R1a binders will be administered in an “effective amount” which is an amount sufficient to cause GABA_{B}R1a modulation. One skilled in the art can readily determine an effective amount of the GABA_{B}R1a binder to be administered to a given subject, by taking into account factors such as the extent of the disease penetration, the age, health and sex of the subject, the route of administration and whether the administration is regional or systemic. Generally, an effective amount of GABA_{B}R1a binders modulating GABA_{B}R1a activity comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of inhibitor can be administered.

### Drug Administration Across the Blood-Brain Barrier

The blood-brain barrier (BBB) is a protective layer of tightly joined cells that lines the blood vessels of the brain which prevents entry of harmful substances (e.g. toxins, infectious agents) and restricts entry of (non-lipid) soluble molecules that are not recognized by specific transport carriers into the brain. This poses a challenge in the delivery of drugs, such as the GABABR1a binders described herein, to the central nervous system or brain in that drugs transported by the blood not necessarily will pass the blood-brain barrier. Several options are nowadays available for delivery of drugs across the BBB (Peschillo et al. 2016, J Neurointervent Surg 8:1078-1082; Miller & O'Callaghan 2017, Metabolism 69:S3-S7; Drapeau & Fortin 2015, Current Cancer Drug Targets 15:752-768).

Drugs can be directly injected into the brain (invasive strategy) or can be directed into the brain after BBB disruption with a pharmacological agent (pharmacologic strategy). Invasive means of BBB disruption are associated with the risk of hemorrhage, infection or damage to diseased and normal brain tissue from the needle or catheter. Direct drug deposition may be improved by the technique of convection-enhanced delivery. Longer term delivery of a therapeutic protein can be achieved by implantation of genetically modified stem cells, by recombinant viral vectors, by means of osmotic pumps, or by means of incorporating the therapeutic drug in a polymer (slow release; can be implanted locally).

Pharmacologic BBB disruption has the drawback of being non-selective and can be associated with unwanted effects on blood pressure and the body's fluid balance. This is circumvented by targeted or selective administration of the pharmacologic BBB disrupting agent. As an example, intra-arterial cerebral infusion of an antibody (bevacizumab) in a brain tumor was demonstrated after osmotic disruption of the BBB with mannitol (Boockvar et al. 2011, J Neurosurg 114:624-632); other agents capable of disrupting the BBB pharmacologically include bradykinin and leukotriene C4 (e.g. via intracarotid infusion; Nakano et al. 1996, Cancer Res 56:4027-4031).

BBB transcytosis and efflux inhibition are other strategies to increase brain uptake of drugs supplied via the blood. Using transferrin or transferrin-receptor antibodies as carrier of a drug is one example of exploiting a natural BBB transcytosis process (Friden et al. 1996, J Pharmacol Exp Ther 278:1491-1498). Exploiting BBB transcytosis for drug delivery is also known as the molecular Trojan horse strategy. Another mechanism underlying BBB, efflux pumps or ATP-binding cassette (ABC) transporters (such as breast cancer resistance protein (BCRP/ABCG2) and P-glycoprotein (Pgp/MDR1/ABCB1)), can be blocked in order to increase uptake of compounds (e.g. Carcaboso et al. 2010, Cancer Res 70:4499-4508). Therapeutic drugs can alternatively be loaded in liposomes to enhance their crossing of the BBB, an approach also known as liposomal Trojan horse strategy.

A more recent and promising avenue for delivering therapeutic drugs to the brain consists of (transient) BBB disruption by means of ultrasound, more particularly focused ultrasound (FUS; Miller et al. 2017, Metabolism 69:S3-S7). Besides being non-invasive, this technique has, often in combination with real-time imaging, the advantage of precise targeting to a diseased area of the brain. Therapeutic drugs can be delivered in e.g. microbubbles e.g. stabilized by an albumin or other protein, a lipid, or a polymer. Therapeutic drugs can alternatively, or in conjunction with microbubbles, be delivered by any other method, and subsequently FUS can enhance local uptake of any compound present in the blood (e.g. Nance et al. 2014, J Control Release 189:123-132). Just one example is that of FUS-assisted delivery of antibodies directed against toxic amyloid-beta peptide with demonstration of reduced pathology in mice (Jordao et al. 2010, PloS One 5:e10549). Microbubbles with a therapeutic drug load can also be induced to burst (hyperthermic effect) in the vicinity of the target cells by means of FUS, and when driven by e.g. a heat shock protein gene promoter, localized temporary expression of a therapeutic protein can be induced by ultrasound hyperthermia (e.g. Lee Titsworth et al. 2014, Anticancer Res 34:565-574). Alternatives for ultrasound to induce the hyperthermia effect are microwaves, laser-induced interstitial thermotherapy, and magnetic nanoparticles (e.g. Lee Titsworth et al. 2014, Anticancer Res 34:565-574).

### Intracellular Drug Administration

Besides the need to cross the BBB, drugs targeting GABA_{B}R1a activity may also need to cross the cellular barrier. One solution to this problem is the use of cell-penetrating proteins or peptides (CPPs). Such peptides enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein.

CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

### Other Definitions

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

“sAPP”, “sAPPα”, “APPα” or “APP” are used herein interchangeably and refer to the secreted amyloid-β precursor protein, more particularly to the extension domain of human sAPPα. The sequence of said extension domain of which the wild-type 17-, 9- and 5-mer peptides of the invention are derived is depicted in SEQ ID No. 2: NVDSADAEEDDSDVWWGGADTDYADGSEDKVVE.

### Examples

The Applicants previously reported that GABABR1a specifically interacts with the sAPP extension domain, more precisely with a 17-amino acid long stretch. Further truncation experiments revealed that a conserved, minimal 9-amino acid sequence within the sAPP 17-mer is sufficient for direct binding to the sushi 1 domain of GABABR1a (Rice et al 2019 Science 363; WO2018/015296). In order to design compounds that modulate the sAPP-GABABR1a interaction and thus GABABR1a activity, the binding of said proteins was studied in further detail.

### Example 1. Variants of the sAPP 9-Mer Bind with Similar Affinity

First, we wondered whether the wild-type sAPP 9-mer peptide would be the optimal sequence in terms of SD1 binding or whether sequence variants of the sAPP 9-mer would have improved binding to the GABABR1a-SD1. Therefore a panel of single and double sAPP 9-mer mutants was generated. An alanine scan was performed for the residues on position 1 to position 7. Based on an ITC assay, these single mutant peptides bound the SD1 with the same or slightly higher Kd (Table 1). Also the double mutants VIB_P04, VIB_P05 and VIB_P19 bound with a similar Kd to SD1 compared to the wild-type 9-mer. We have also tested the effect of the D-isomer of the amino residues on position 1 to 4, in single or multiple combinations. None of the mutations had strong effects on the measured Kd (Table 1). We therefore conclude that the wild-type sAPP 9-mer is already optimized but that many variants bind as good to SD1 and thus will modulate GABABR1a in a similar manner.

TABLE 1
Binding properties of the wild-type sAPP 17-mer and 9-mer as well as 9-mer mutants
based on the ITC assay.

| Peptide | | Residue Position | | | | | | | | | ITC |
|---|---|---|---|---|---|---|---|---|---|---|---|
| VIB # | Sequence | 1 | 2 | 3 | 4 | 5 | 6 | 7 | 8 | 9 | Kd μM |
| 17-mer | DDSDVWWGGADTDYADG | | | | | | | | | | 0.81 |
| 9-mer | DDSDVWWGG | D | D | S | D | V | W | W | G | G | 3.00 |
| VIB_P12 | ADSDVWWGG | A | D | S | D | V | W | W | G | G | 7.58 |
| VIB_P01 | DASDVWWGG | D | A | S | D | V | W | W | G | G | 12.17 |
| VIB_P02 | DDADVWWGG | D | D | A | D | V | W | W | G | G | 3.68 |
| VIB_P03 | DDSAVWWGG | D | D | S | A | V | W | W | G | G | 10.01 |
| VIB_P16 | DDSDAWWGG | D | D | S | D | A | W | W | G | G | |
| VIB_P17 | DDSDVAWGG | D | D | S | D | V | A | W | G | G | |
| VIB_P18 | DDSDVWAGG | D | D | S | D | V | W | A | G | G | |
| VIB_P19 | AASDVWWGG | A | A | S | D | V | W | W | G | G | 18.05 |
| VIB_P04 | DASAVWWGG | D | A | S | A | V | W | W | G | G | 5.85 |
| VIB_P05 | ADSAVWWGG | A | D | S | A | V | W | W | G | G | |
| VIB_P08 | {D-ASP}DSDVWWGG | d | D | S | D | V | W | W | G | G | 6.45 |
| VIB_P09 | D{D-ASP}SDVWWGG | D | d | S | D | V | W | W | G | G | 11.03 |
| VIB_P10 | DD{D-SER}DVWWGG | D | D | s | D | V | W | W | G | G | 8.47 |
| VIB_P11 | DDS{D-ASP}VWWGG | D | D | S | d | V | W | W | G | G | 6.21 |
| VIB_P07 | {D-ASP}{D-ASP}{D-SER}DVWWGG | d | d | s | D | V | W | W | G | G | 11.00 |
| VIB_P20 | DDSDVWWG | D | D | S | D | V | W | W | G | | 1.95 |
| VIB_P33 | DVWWG | | | | D | V | W | W | G | | 9.09 |
| VIB_P06 | DDSDVWW | D | D | S | D | V | W | W | | | 25.84 |

### Example 2. sAPP 5-Mer can Still Bind to GABABR1a

In the mutagenesis experiment of Example 1 we also included an 8-mer lacking position 9. We surprisingly found that the 8-mer DDSDVWWG has an improved Kd of 1.95 μM compared to 3 μM of the 9-mer (Table 1). This suggests that the 9-mer can be truncated further. We therefore removed the first 3 residues as well and surprisingly found that the DVWWG 5-mer (herein referred also as VIB-P33) could still bind the sushi domain 1 of GABA_{B}R1a with a Kd of 9.09 (Table 1).

### Example 3. A Fluorescence Polarization Assay to Screen for GABA_{B}R1a Binding Peptides

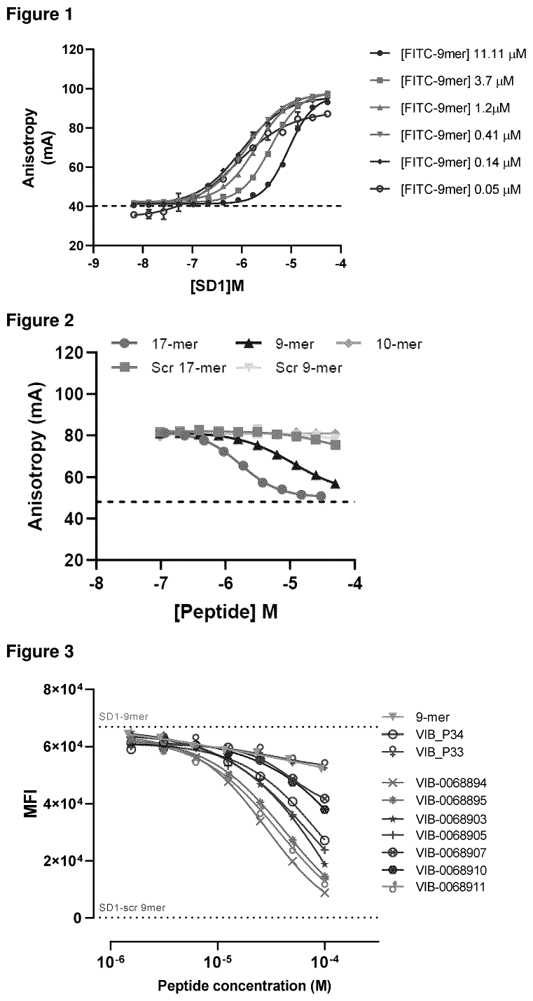

Intrigued by the surprising results of the 5-mer, it was decided to set up a medium- to high-throughput screen to optimize the 5-mer sequence. Several assays were tested to find a reproducible, robust and cost-effective assay that is also suitable for high-throughput screening. A fluorescence polarization (FP) assay was selected. FP measurements are well-known for receptor binding assays. The assay is based on the rotational movement of fluorescently labelled molecules in solution. Unbound molecules rotate rapidly, are therefore randomly orientated prior to light emission and hence show a low polarization value. However, if the rotation of a fluorescently labelled molecule is slowed down because it binds to a large complex, it shows a high polarization value. The FP assay was optimized using the FITC labelled sAPP 9-mer (FIG. 1).

To screen for GABA_{B}R1a binders using unlabelled test compounds, the assay was turned into a competition assay using FITC-9mer. Hence, binding of a test compound is detected by the decrease in FP signal. To validate this inhibitor assay, the IC50 values were determined of the 17-mer, 9-mer, 5-mer as well as those from 3 negative controls: 10-mer, scrambled 17-mer and a scrambled 9-mer (FIG. 2). With the 17-, 9- and 5-mer, IC₅₀ values could be obtained of 1.7, 11 and 39 μM respectively, while the 10-mer and scrambled peptides did not bind.

### Example 4. Novel and Potent 5-Mer GABABR1a Binders

Next, 35 variants of the DVWWG 5-mer peptide containing natural residues were tested in the above described FP assay. The peptides were tested at a concentration of 50 μM. Interestingly, 11 additional peptides could be identified with IC₅₀ value of less than 100 μM. Surprisingly, 5 peptides were found that were more potent than the wild type sAPP 5-mer VIB_P33 (Table 2).

These results demonstrate that the tryptophane (W) at position 4 is a key residue. A limited number of alternative residues are allowed on the other positions without a strong penalty on the IC₅₀ values. Although aspartate (D) on position 1 can be replaced by asparagine (N) or glycine (G) but also by serine (S) or proline (P), D is preferred for optimal binding to SD1. A similar situation occurs at position 3: the tryptophane (W) can be replaced by tyrosine (Y), histidine (H) or phenylalanine (F), but W is preferred. Binding of the sAPP 5-mer to SD1 can be optimized by replacing valine (V) at position 2 to isoleucine (I). Finally, by replacing the glycine (G) at position 5 by serine (S) the IC50 values are reduced by half. Interestingly, a synergistic effect could be seen when both V at position 2 was replaced by I and G on position 5 by S (VIB_P34).

TABLE 2
Binding properties and ex vivo activity of the wild-type 5-mer (VIB_P33) and 5-mer mutants.

| | | | | | | In vitro assay | | | Cell-based | |
|---|---|---|---|---|---|---|---|---|---|---|
| | Residue Position | | | | | ITC | FP | | Ex vivo assay | |
| Peptide VIB # | 4 | 5 | 6 | 7 | 8 | Kd μM | % inhibition 50 μM | IC₅₀ μM | 1 μM | 10 μM |
| VIB_P33 | D | V | W | W | G | 9.09 | 66.00 | 39.32 | | |
| VIB_P34 | D | I | W | W | S | 1.02 | 81.87 | 7.64 | Active | Active |
| VIB_P39 | D | V | W | W | S | 4.45 | 85.52 | 13.95 | | |
| VIB_P29 | D | I | W | W | G | 1.89 | 75.00 | 16.51 | Active | |
| VIB_P43 | D | I | Y | W | S | 4.3 | 48.59 | 21.18 | | Active |
| VIB_P37 | D | I | H | W | S | 8.26 | 20.00 | 35.55 | | |
| VIB_P66 | N | I | W | W | S | 12.36 | 66.44 | 40.83 | | |
| VIB_P35 | D | I | F | W | S | 5.71 | 55.85 | 51.53 | | |
| VIB_P42 | D | I | Y | W | G | 12.72 | 47.94 | 62.31 | | |
| VIB_P58 | N | F | Y | W | S | | −7.00 | 62.90 | | |
| VIB_P65 | N | I | W | W | G | 14.49 | 41.40 | 76.07 | | |
| VIB_P53 | D | V | Y | W | G | | 33.47 | 79.24 | | |
| VIB_P60 | G | V | Y | W | S | | 47.52 | 84.30 | | |
| VIB_P48 | N | V | W | W | S | | 33.55 | 93.71 | | |
| VIB_P47 | G | I | Y | W | S | 7.03 | 53.36 | 217.20 | | Active |
| VIB_P22 | D | V | F | W | S | 14.41 | | | | |
| VIB_P24 | D | V | Y | W | S | 15.77 | | | | |
| VIB_P36 | D | I | H | W | G | 18.45 | 25.48 | | | |
| VIB_P56 | N | V | W | W | G | 22.9 | 17.94 | | | |
| VIB_P41 | S | V | Y | W | S | 23.6 | 5.28 | | | |
| VIB_P68 | N | I | Y | W | G | 25 | 15.87 | | | |
| VIB_P23 | D | V | H | W | S | 25.06 | | | | |
| VIB_P45 | P | I | H | W | S | 38.02 | | | | |
| VIB_P63 | N | I | Y | W | S | 49.5 | 19.41 | | | |
| VIB_P49 | P | I | Y | W | S | 65.83 | 24.96 | | | |
| VIB_P25 | D | V | F | W | G | 72.99 | | | | |
| VIB_P44 | P | V | H | M | S | | −7.00 | 185.50 | | |

In summary, 13 peptides consisting of 5 natural amino acids were identified that inhibited the sAPP 9-mer binding to the GABABR1a-SD1 in the FP assay with at least 30% and with an IC₅₀ of less than 100 μM. The peptides have the consensus sequence X1 X2 X3 W X5, wherein X1 can be D, N, G, S or P; X2 can be V or 1; X3 can be W, F, H or Y; X5 can be G or S.

### Example 5. Non-Natural GABABR1a Binders

Based on structural information of DIYWS-SD1 and DIWWS-SD1 co-crystals, 48 peptides containing non-natural residues were manually designed and checked for binding to the GABABR1a SD1 in the FP assay at a concentration of 50 μM. These non-natural peptides were categorized based on the IC50 values obtained in the FP assay (Table 3). Peptides with an IC50 of more than 100 μM in the FP assay are not listed. Interestingly, several non-natural peptides were identified that bound 7× more potently to SD1 compared to the wild-type 5-mer VIB-P33 (Table 2 & 3), while others (e.g. VIB-0068911 and VIB-0068894) bound as good as the 17-mer (Table 1 & 3). The listed non-natural 5-mers all share D-aspartate was on position 1 (residue position 4 of the 9-mer), L-isoleucine on position 2 (residue position 5 of the 9-mer), L-tryptophane on position 4 (residue position 7 of the 9-mer) and L-serine on position 5 (residue position 8 of the 9-mer), while position 3 is selected from the list consisting of L-tyrosine, isoethylmethyl-benzene, 6-chloro-3-methyl-1H-indole, methylcyclohexane, ethylcyclohexane, 2-naphthalene, ethylbenzene, 1,1-difluoro-4-cyclohexyl, 4-methyl-1-methoxy-2-methylbenzene, 1-chloro-4-methylbenzene, 4-methylphenyl-methanol, 3-methylbenzoic acid and 4-methylaniline.

TABLE 3
Binding properties of non-natural 5-mer APP peptides.

| | | | | | | In-vitro system | | | | |
|---|---|---|---|---|---|---|---|---|---|---|
| | | | | | | ITC | FP | | Flow-Cytometry | |
| | Residue Position | | | | | Kd | % inhibition | IC₅₀ | % inhibition | IC₅₀ |
| Peptide VIB # | 4 | 5 | 6 | 7 | 8 | μM | 50 μM | μM | 100 μM | μM |
| VIB-0068911 | d | I | F72 | W | S | 0.54 | 98.09 | 6.54 | 89.99 | 37.95 |
| VIB-0068894 | d | I | F126 | W | S | 0.34 | 96.84 | 7.12 | 84.20 | 28.67 |
| VIB-0068905 | d | I | F15 | W | S | 0.25 | 94.80 | 8.11 | 68.27 | 78.76 |
| VIB-0068903 | d | I | F141 | W | S | 0.93 | 93.90 | 8.22 | 76.03 | 154.00 |
| VIB-0068895 | d | I | F195 | W | S | 1.39 | 96.27 | 11.13 | 77.00 | 43.30 |
| VIB-0068910 | d | I | F70 | W | S | 2.90 | 88.62 | 11.93 | 54.17 | |
| VIB-0068907 | d | I | F182 | W | S | 2.47 | 86.17 | 12.64 | 47.18 | 49.17 |
| VIB-0068906 | d | I | F158 | W | S | | 70.77 | 20.93 | | |
| VIB-0068912 | d | I | F86 | W | S | | 66.23 | 32.70 | | |
| VIB-0068896 | d | I | F44 | W | S | 7.46 | 56.62 | 47.98 | | |
| VIB-0068908 | d | I | F50 | W | S | | 53.13 | 53.80 | | |
| VIB-0068909 | d | I | F57 | W | S | | 44.25 | 88.29 | | |
| VIB-0068904 | d | I | F149 | W | S | 1.02 | 26.68 | | | |
| VIB-0068902 | d | I | Y | W | S | | 76.77 | 11.64 | | |
| VIB-0068870 | d | I | Y | W | S* | | 34.58 | 16.54 | | |
| VIB-0068875 | d | I | Y | W | S** | | 41.77 | 71.19 | | |
| VIB-0068884 | d* | I | Y | W | S | | 41.46 | 100.30 | | |
| VIB-0068914 | d | I | Y | Y | S | | 72.40 | 25.11 | | |

S* = L-serine coupled to C-N-(1,3,4-thiadiazol-2-yl)amide;
S** = L-serine coupled to C-N-(2-(4-methylpiperazin-1-yl)-2-oxoethyl)amide;
d* = D-aspartic acid coupled to (1-aminocyclobutyl)N-methanone,
F72 = (isoethylmethyl)benzene,
F126 = 6-chloro-3-methyl-1H-indole,
F15 = methylcyclohexane,
F141 = ethylcyclohexane,
F195 = 2-naphtalene,
F70 = ethylbenzene,
F182 = 1,1-difluoro-4-cyclohexyl,
F158 = 4-methyl-1-methoxy-2-methylbenzene,
F86 = 1-chloro-4-methylbenzene,
F44 = (4-methylphenyl)methanol,
F50 = 3-methylbenzoic acid,
F57 = 4-methylaniline,
F149 = N-ethyl-tryptophan

We also tested variants of the DIYWS 5-mer (VIB_P43) described in Example 4. While mutating the L-Aspartic acid to the D-stereoisomer did not have an effect on the binding properties of the wild-type 9-mer (see Table 1), it reduced the IC₅₀ value of the DIYWS 5-mer from 21.18 to 11.64 μM (Table 3). Mutating the L-Tryptophan to L-Tyrosine on position 4 (residue position 7 of the 9-mer) nullified the decrease in IC50. Finally, it was tested whether adding chemical groups to the serine or the D-aspartic acid have an effect on the binding properties of the dIYWS peptide (Table 3).

### Example 7. Confirmation of GABABR1a Binders by ITC and Cell-Based Assays

The most potent natural and non-natural peptides in the FP assay were confirmed by ITC potency determination. In contrast to the FP assay, the isothermal titration calorimetry (ITC) assay is a direct binding assay. The FP results could be confirmed as several peptides performed as good as the wild-type 17-mer while others were 20× more potent than the wild-type 5-mer (VIB-P33) (Table 2 & 3).

Next, the top ranking compounds were tested in a cell-based binding assay. HEK293 cells were transformed to express the ectodomain of GABABR1a comprising SD1. In this cell-based assay, SD1 is expressed on the GABABR1a ectodomain and can adopt a more physiological conformation, compared to the purified SD1 protein used in the primary assay (FP assay). Before testing the active compounds, it was confirmed that 9-mer (tagged with Biotin) binds specifically to cells expressing GABABR1a-SD1 and not GABABR1b. The 9-mer scramble peptide did not show any binding.

All compounds tested were able to inhibit the binding of the 9-mer to the GABABR1a-SD1. The most potent peptides could reduce the binding of the 9-mer with more than 85% (Table 3; FIG. 3).

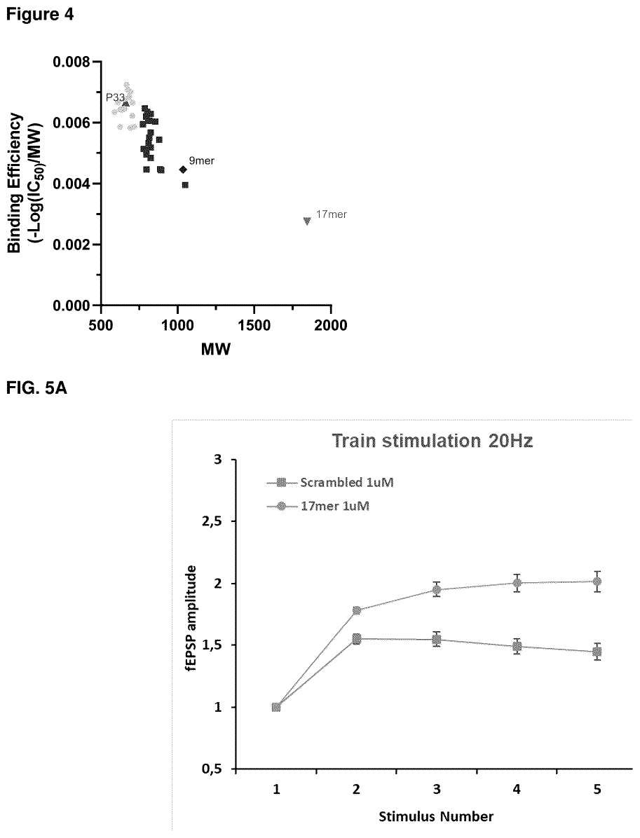

Finally, we also determined a binding efficiency index for the most interesting natural and non-natural peptides. We therefore normalized the interaction affinity with the molecular weight (MW). The binding efficiency index thus visualizes the relationship between affinity (Kd value measured by the ITC assay) and molecular size of the compounds. As can be appreciated from FIG. 4, all listed compounds performed significantly better compared to the wild-type sAPP 17-mer.

### Example 8. Modulation of Synaptic Activity

The effect of a selection of GABA_{B}R1a-SD1 binders on synaptic transmission was tested in an ex vivo experiment using acute mouse hippocampal slices comprising an intact circuit of CA3-CA1 Schaffer collateral (SC) synapses. In short, a burst of 5 stimuli at 20 Hz was applied to Schaffer collaterals to induce short-term facilitation, which inversely correlates with the probability of neurotransmitter release. We previously showed that application of 1 μM sAPPα recombinant protein increases facilitation compared to controls in this assay and demonstrated that sAPPα controls synaptic vesicle release in a GABA_{B}R-dependent manner at this synapse (Rice et al 2019 Science).

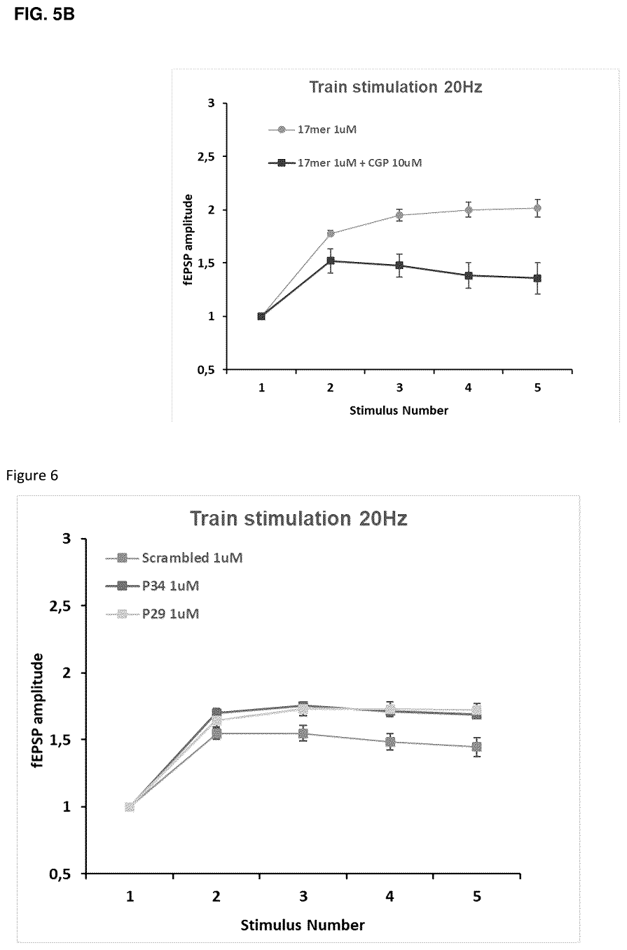

We first repeated this experiment using the sAPP 17-mer instead of sAPPα recombinant protein. From FIG. 5A it can be appreciated that administration of 1 μM sAPP 17-mer to acute hippocampal slices increased the facilitation compared to the scrambled control peptide, indicating a decreased synaptic vesicle release probability. Furthermore, preincubation with the GABA_{B}R antagonist CGP-54626 (10 μM) blocked the sAPP 17-mer induced increase in facilitation, indicating that the effect is GABA_{B}R-dependent (FIG. 5B).

We next tested the effect of sAPP 5-mer peptides on facilitation (FIG. 6). A similar statistically significantly increase in facilitation compared to the scrambled control could be seen upon adding the 5-mer P34 (1 μM) as well as P29 (1 μM). This demonstrates that these 5-mers suppress synaptic vesicle release ex vivo.

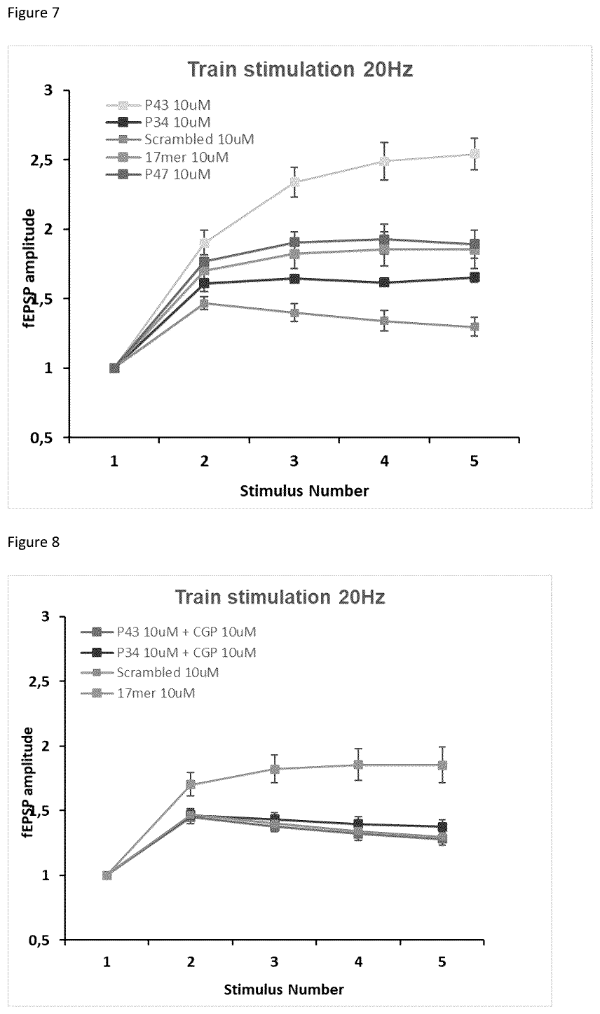

We next tested the 5-mers P43, P34 and P47 at a 10 μM concentration, considering their lower binding affinity to SD1 as determined by ITC (Table 2). P43, P34 and P47, as well the 17-mer (all at 10 μM) as positive control, all showed a statistically significant increase in short-term facilitation compared to the scrambled negative control at 10 μM (FIG. 7).

The P34 and P43 5-mers were also tested in hippocampal slices preincubated with the GABA_{B}R antagonist CGP-54626 (FIG. 8). The previously observed increase in short-term facilitation could not be detected anymore, demonstrating that the DIWWS and DIYWS peptides control vesicle release at SC synapses in a GABA_{B}R-dependent manner.

### Experimental Details

### Recombinant Sushi1 Expression and Purification

For biophysical and structural biology purposes the Sushi1 protein was expressed in a bacterial expression system. The synthetic gene encoding for residues 26-96 of the Sushi1 protein was cloned into a pFloat-SUMO vector, generating a His-tagged SUMO-Sushi1 fusion protein. The construct also contained a 3C protease cleavage site to remove the His-SUMO-tag. The pFloat-SUMO-Sushi1 plasmid was transformed in BL21(DE3) cells and plated on kanamycin (100 ag/ml) containing LB agar plates. A small LB culture, supplemented with 100 ag/ml kanamycin, was inoculated with a single colony of BL21(DE3)(pFloat-SUMO-Sushi1) and grown overnight at 37° C. 1 liter LB cultures were subsequently inoculated with 20 ml of this preculture and grown at 37° C. until OD₆₀₀ reached 0.8. At this point protein expression was induced using 0.5 mM isopropyl β-D-1-thiogalactopyranoside (IPTG). Cells were incubated further overnight at 20° C. and subsequently harvested by centrifugation (Beckman rotor 8.1000, 5000 rpm, 15 min, 4C). The pellet was resuspended in 20 mM Tris pH 7.5, 500 mM NaCl, 10 mM imidazole, 5 mM β-mercaptoethanol, 0.1 mg/mL 4-(2-aminoethyl) benzenesulfonyl fluoride hydrochloride (AEBSF), 1 μg/mL leupeptine, 50 μg/mL DNasel and 20 mM MgCl₂. The cells were lysed using a French press (Constant Systems) at 20 kpsi and the cell debris was removed by centrifugation. The cell lysate was loaded on a Ni-sepharose FF HiLoad column (GE Healthcare), equilibrated in 20 mM Tris pH 7.5, 500 mM NaCl, 10 mM imidazole, 5 mM 9-mercaptoethanol. The bound proteins were eluted using a linear gradient to 500 mM imidazole. Fractions containing the His-SUMO-Sushi1 protein were pooled and dialysed overnight to 20 mM Tris pH 7.5, 150 mM NaCl, while cleaving with 3C protease. The cleaved sample was loaded again on a Ni-sepharose FF HiLoad column, equilibrated in the same buffer. The FT, containing the Sushi1 protein, was concentrated and applied to a BioRad S100 16/60 size exclusion column, equilibrated in 50 mM KPi buffer pH 6.0, 50 mM NaCl.

For expression of ¹³C/N1⁵ labelled Sushi1 cultures were grown in 500 mL Min9 medium (6.8 g/L Na₂HPO₄, 3 g/L KH₂PO₄, 1 g/l NaCl) supplemented with 50 mg/L EDTA, 0.2 mg/L H₃BO₃, 3 mg/L CuCl₂-2H₂O, 7 mg/L ZnSO₄-7H₂O, 8 mg/L CoCl₂-6H₂O, 12 mg/mL MnCl₂-4H₂O, 60 mg/L FeSO₄-7H₂O, 2 mM MgSO₄, 0.2 mM CaCl₂, 2.5 g/L ¹³C glucose, 1 g/L¹⁵NH₄Cl and 50 μg/ml kanamycin (inoculated with 1 ml of LB preculture) instead of LB. All other steps of the expression and purification protocol were unchanged.

### Isothermal Titration Calorimetry (ITC) Measurements

The Sushi-1 protein was dialysed overnight to PBS buffer and diluted to 30 μM in the same buffer. The peptides were resuspended in PBS at a stock concentration of 3 mM and diluted further in the same buffer to 300 μM. ITC experiments were performed on a Microcal ITC200 device. Titrations comprised 26×1.5 μL injections of peptide into protein, with 90 s intervals. An initial injection of ligand (0.5 μL) was made and discarded during data analysis. The data were fitted to a single binding site model using the Microcal LLC ITC₂₀₀. Original software provided by the manufacturer.

### Multi-Electrode Array Electrophysiology

Recordings were done in C57BL/6J mice (2 months old). For tissue preparation, mice were anesthetized with isoflurane and rapidly decapitated to prepare acute 300 μm-thick parasagittal brain slices on a Leica VT1200 vibratome. Slicing was performed in cold sucrose based cutting solution (ACSF) that consisted of (in mM): 87 NaCl, 2.5 KCl, 1.25 NaH2PO4, 10 glucose, 25 NaHCO₃, 0.5 CaCl2, 7 MgCl2, 75 sucrose, 1 kynurenic acid, 5 ascorbic acid, 3 pyruvic acid (pH 7.4 with 5% CO2/95% O2). Slices were allowed to recover at 34° C. for 35 min, and subsequently maintained at room temperature for at least 30 min before use. Before recordings, slices were preincubated in artificial cerebrospinal fluid (ACSF, 119 mM NaCl, 2.5 mM KCl, 1 mM NaH2PO4, 11 mM glucose, 26 mM NaHCO₃, 4 mM MgCl2 and 4 mM CaCl2, pH 7.4) containing peptide of interest or scrambled/control peptide (1 or 10 uM). Preincubation was performed in the multielectrode array recording chamber (60MEA200/30iR-Ti-gr, Multichannel Systems) for 60 min at 32° C., using custom-made local carbogenation rings (5% CO2/95% 02). Field excitatory post-synaptic potentials (fEPSPs) were recorded from Schaffer collateral-CA1 synapses by stimulating and recording from the appropriate (visually identified) electrodes (MEA-2100, Multichannel Systems). Input-output curves were recorded for each slice by applying single-stimuli ranging from 500 to 2750 mV with 250 mV increments. Stimulus strength that corresponds to 35% of maximal response in the input-output curve was used for following recordings. Train stimulations of 10, 20 and 50 Hz (5 stimuli per train) were recorded with 10 minutes intervals. Recordings were processed and analysed using Multi Channel Experimenter software (Multi Channel Systems).

### Fluorescence Polarization Assay

To determine if the peptides of this application were able to disrupt the SD1-9-mer complex, each peptide (in single point or dose-response curve) tested was incubate with SD1 protein for 1 h at RT. After this initially incubation, FITC-9mer was added to the previous mix and incubated during 2 h at RT. The signal intensity was measure in an appropriate plate reader using the following settings: Number of flashes per well: 200; Excitation: F482-16; Dichroic filter: FLP 504; Emission A F530-40; Gain A: 738, Gain B: 736. The assay was conducted in a 384-well plate.

### Flow-Cytometry Assay

HEK293 were transfected with plasmids encoding the ectodomain of GABABR1a (as in Rice et al., 2019), carrying a His-tag at the N-terminal. After 24 h of expression, cells were harvested and adjusted to a concentration of 1×106 cells/mL in ice-cold 2% FBS/PBS. Incubation with each peptide (in dose-response curve) was carried out at 4° C. for 1 h, followed by incubation with the biotion-9mer (10 μM) peptide during 30 min. Detection antibodies recognizing either His protein or biotin were added for a period of 30 min. The assay plate containing the cells was loaded into the Attune NxT Cytometer, parameters were adjusted accordingly with the cell type and signal intensity. In each experiment, positive and negative control were added to the plate.

1. A GABA_{B}R1a binding peptide comprising the sequence X₁X₂X₃X₄X₅, wherein
X₁ is D, N, G, P or S;
X₂ is V or I;
X₃ is W, F, Y or H;
X₄ is W or Y; and
X₅ is G or S,
or a peptidomimetic thereof, wherein the GABA_{B}R1a binding peptide or peptidomimetic thereof has a length of between 5 and 17 amino acids and wherein the GABA_{B}R1a binding peptide or peptidomimetic thereof does not comprise DDSDVWWGG.

2. The GABA_{B}R1a binding peptide or peptidomimetic thereof of claim 1, wherein X₄ is W.

3. The GABA_{B}R1a binding peptide or peptidomimetic thereof of claim 1, wherein the peptide is selected from the group consisting of DVWWG, DVWWS, DIWWS, DIWWG, DIFWS, DIYWS, DIYWG, GVYWS, NIWWG, NVWWS, and DVYWG.

4. The GABA_{B}R1a binding peptide or peptidomimetic thereof of claim 1, wherein X₁ is D, X₂ is I, X₄ is W, X₅ is S, and wherein X₃ is selected from the group consisting of isoethylmethyl-benzene, 6-chloro-3-methyl-1H-indole, methylcyclohexane, ethylcyclohexane, 2-naphthalene, ethylbenzene, 1,1-difluoro-4-cyclohexyl, 4-methyl-1-methoxy-2-methylbenzene, 1-chloro-4-methylbenzene, 4-methylphenyl-methanol, 3-methylbenzoic acid, N-ethyl-tryptophan, and 4-methylaniline.

5. The GABA_{B}R1a binding peptide or peptidomimetic thereof of claim 4, wherein the GABA_{B}R1a binding peptide or peptidomimetic thereof is selected from the group consisting of VIB-0068911-011, VIB-0068894-001, VIB-0068905-001, VIB-0068903-001, VIB-0068895-001, VIB-0068902-001, VIB-0068910-001, VIB-0068907-001, VIB-0068870-001, VIB-0068906-001, VIB-0068914-001, and VIB-0068912-001.

6. The GABA_{B}R1a binding peptide or peptidomimetic thereof of claim 1, wherein the GABA_{B}R1a binding peptide or peptidomimetic thereof is comprised in a pharmaceutical composition.

7. (canceled)

8. A method of treating a subject, the method comprising:
administering to the subject the GABA_{B}R1a binding peptide or peptidomimetic thereof of claim 1, wherein the subject suffers from one or more of cognitive impairment, anxiety, depression, epilepsy, dystonia, CMT, Alzheimer's disease, neuropathic pain, narcolepsy, and spasticity.
